(19) United States
(12) Patent Application Publication
(10) Pub. No.: US 2016/0345902 A1
(43) Pub. Date: Dec. 1, 2016

(54) SYSTEM TO MEASURE FOOT FUNCTION

(71) Applicant: BEND TECH, LLC, Draper, UT (US)

(72) Inventors: JULES A. DEGREEF, DRAPER, UT (US); DAVID B. BECK, DRAPER, UT (US); JOHN M. FORST, WEST JORDAN, UT (US); JOSEPH A. HARRIS, SANDY, UT (US)

(21) Appl. No.: 15/173,623

(22) Filed: Jun. 4, 2016

Related U.S. Application Data
(63) Continuation-in-part of application No. 12/604,951, filed on Oct. 23, 2009, now Pat. No. 9,357,947.

(51) Int. Cl. A61B 5/00; A61B 5/103

(52) U.S. Cl. CPC A61B 5/6807; A61B 5/742; A61B 5/7475; A61B 5/1038; A61B 5/0022; A61B 5/7405; A61B 2560/0475; A61B 2560/0214; A61B 2562/0219; A61B 2562/0247

(57) ABSTRACT

A system for measuring the functioning of the feet of a user includes an insert having an arrangement of sensors positioned in footwear to sense the pressure or deflection of the sensors by a user engaged in activity like walking, running, or playing a sport (e.g., golf; tennis, football, basketball). The sensor arrangement for each foot includes sensors at multiple locations under or proximate a foot which sensors each predictably vary in resistance upon application of a force thereto. The sensors supply analog signals to a circuit which in turn sends signals reflective of the force through an A-D converter for transmission wirelessly by, for example, a wireless Bluetooth device. The digitalized signals reflective of deflection are collected and sent to the remote control device to compute and display images reflective of the force of the foot experienced at each of the multiple locations. An accelerometer may also be combined proximate to or attached to the insert to supply signals reflective of the acceleration and which can be used to calculate and display other information including acceleration. A wireless charging structure is included to obtain power wirelessly and to supply power to the insert.

### RELATED APPLICATIONS

This application is a continuation-in-part application of U.S. patent application Ser. No. 12/604,951, filed Oct. 23, 2009, the contents of which are incorporated herein by reference.

### BACKGROUND OF THE INVENTION

### Field of the Invention

This application relates to a system used to measure the functioning of a user's feet when involved in activity and more particularly includes a device for positioning between the support surface of footwear and the foot of a user supported in or on the footwear to sense the functioning of the foot as the user moves that foot. More particularly, this application relates to a sensor arrangement that is positioned on or above the support surface of an item of footwear that detects the force exerted by at least one portion of the user's foot with the user positioned on and supported in an upright position on the footwear and also to sense the velocity and acceleration of the user's foot.

### State of the Art

When standing upright, a human is typically supported by or deemed to be standing on his or her two feet. It is generally accepted that each foot has three areas of support, namely the heel, the ball (behind the big toe) and the outside (behind the little toe). It is also understood that many people have legs of different length and feet of different size. In turn, the weight of an upright person may not be evenly distributed between left and right legs and/or, in turn, between left and right feet. In addition, the feet of a user may be oriented so that the three areas of support are not in a plane. In turn, the weight of the user is borne unevenly between the three points of support.

A human or other biped can engage in a wide variety of activity that involves operation of the one or both of the user's feet. That is, a user can engage in walking, logging and running. In sports, the user is typically involved in one of these activities in one form or in combinations. For example, sports that involve movement of the feet directly and indirectly include, but are in no way limited to, track and field, skiing, skating, bowling, soccer, football, basketball, hockey, lacrosse, golf, baseball, tennis, ping pong, squash and fencing. In effect, all such activity involves movement of the body and/or feet in a way that the weight or force on the feet and, in turn, on the points of support will vary.

For many reasons it is desirable to know the relative distribution of forces between each of the points of support of a foot, the distribution of weight between feet, and the weight on each foot while standing and while moving. Devices to effectively measure the weight on each of the points of support and the distribution of weight between feet as well as to measure the forces or weight on each foot are unknown. At the same time, it may be desired to know the velocity of the foot and the acceleration of the foot as it is being moved by the user in one direction or another to evaluate the movement.

### SUMMARY OF THE INVENTION

A sensing system includes an insert for placement in an item of footwear under the foot of a user. The insert has at least one sensor positioned to sense the deflection of the support surface affected by the user's foot when the user is upright and either stationary or moving. The sensor is configured to transmit or supply detection signals each reflective of the deflection induced by the user's foot.

A circuit is connected to at least one sensor to receive said detection signals from the at least one sensor. The circuit includes an analog to digital converter to convert said detection signals to digital deflection signals. The circuit also has a processor to process the digital detection signals and generate sensed deflection signals reflective of the detection signals of the at least one sensor and to supply a sensed deflection signal to a transmitter configured to wirelessly transmit the sensed detection signals. The circuit also has power storage structure to receive and store power and to supply electrical power to its components.

The system also includes a flash memory connected to receive the digital detection signals and to the processor which computes the deflection of the sensor(s). The system also includes a control device to wirelessly receive the signals from the transmitter and to display a perceivable image reflective of said deflection of said sensor. The image may show units of deflection, three, distance or some other data that can be calculated from the deflection signal. A power supply means is also provided to supply power to the sensor, the converter means, the memory means and the computer means.

In preferred arrangements, each of a plurality of sensors is positioned proximate to different support points of the foot. In more preferred arrangements, three sensors are positioned proximate to different support points of the foot.

In preferred constructions, the sensor is a substrate with a resistance material deposited thereon. The resistance material is of the type that predictably changes its electrical resistance upon deflection such as an epoxy and carbon composition. In preferred arrangements each sensor is comprised of elongated sensors with enough length to capture the movement of each of the desired support areas of the foot. Each sensor with a connector attached to the opposite ends of the resistance material.

In a more preferred arrangement the control device has controls to select functions to operate the control device to present the user with selected images and selected data.

In a highly preferred arrangement, the system includes an accelerometer mounted on the insert. The accelerometer is configured to sense the acceleration of the insert as it is moved by the user. The accelerometer is connected to supply an acceleration signal to the converter means which, in turn, supplies a digital acceleration preferably through the transmitter to the control device.

In preferred structures the insert may be a pad that is, in effect, an insole that can be inserted into a shoe of the user. Of course, it should he understood that in preferred configurations, the sensors along with the power supply and the circuit are encapsulated so they are water resistant yet pliable.

### DESCRIPTION OF THE DRAWINGS

In the drawings, which illustrate what are presently regarded as the preferred embodiments of the systems and devices that have been disclosed;

FIG. 1 is a perspective view of an insert for use with a system;

FIG. 2 is a perspective view of the bottom of a foot;

FIG. 3 is a cross section of an insert of a disclosed structure in a shoe;

FIGS. 4A and 4B are a block diagram of a sensing system of the disclosed insert;

FIG. 5 is a perspective depiction of a belt mounted device for use as part of the present system as disclosed;

FIG. 6 is a chassis to contain system components;

FIG. 6A is a top view of an alternate and preferred insert for use with the disclosed system;

FIG. 7 is a top view of a power supply and the circuit of the disclosed system;

FIG. 8 is a side view depicting the insert of FIG. 6A along section lines 8-8;

FIG. 9 is a top view of a detector used in the insert of FIG. 6A;

FIG. 10 is a side view of the detector of FIG. 9;

FIG. 11 is a block diagram of the components shown in FIG. 6A;

FIG. 12 is a face view of a control device with inserts of the disclosed system; and

FIG. 13 is a simplified block diagram of the operation of the control device seen in FIG. 12.

### DESCRIPTION OF THE ILLUSTRATED EMBODIMENTS

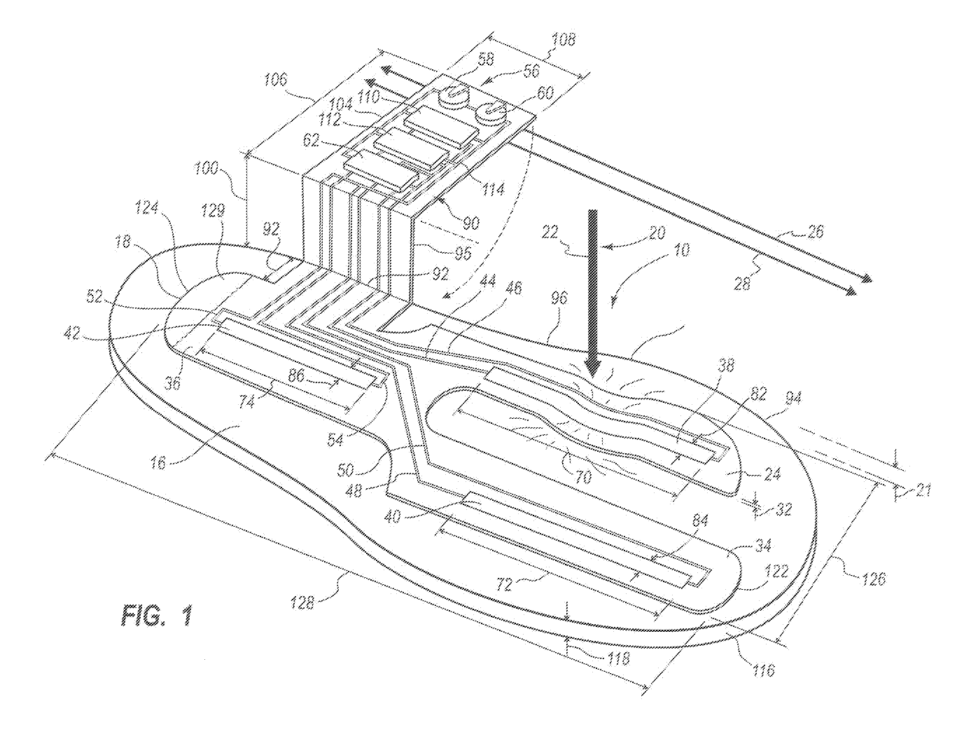

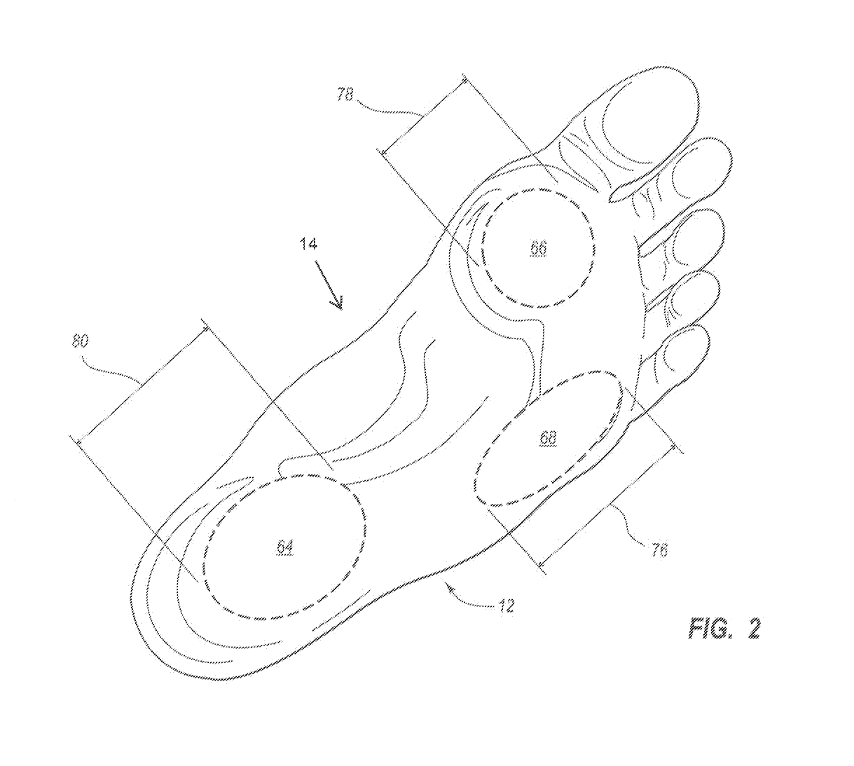

In the drawings, FIG. 1 depicts in perspective an insert 10 sized for placement on a support surface of an item of footwear. As used herein, footwear includes anything that may be worn by the user on one foot or both of his or her feet and has some form of support structure between the bottom 12 of the user's foot 14 (FIG. 2) and a support surface 16 (FIG. 1). Thus, footwear as contemplated herein includes virtually all structures, devices, items and/or things by whatever name that are placed on a foot or the feet of a user including shoes, boots, and sandals, In preferred applications, the support surfaces that support the user's foot are deflectable in some fashion as discussed more fully hereinafter.

The user discussed in connection with the preferred embodiments is a typical human or hominid. The user may be male or female and of any age so long as the user is able to stand upright and walk. Further, it is within contemplation that the user may include quadrupeds and other hominoids such as apes. Further, it should be understood that the principles of the invention apply to both feet even though FIG. 2 shows the bottom 12 only of the foot 14 Which is the left foot of a user. The right foot has not been illustrated for simplicity.

The insert 10 of FIG. 1 has a substrate or base 18 that is made of an electrically insulating material. At the same time, it is durable and long wearing while being flexible and elastically deformable. Various polymers such as polyimide, polycarbonate and polyesters are believed to be particularly suitable. For example, E. I. DuPontand de NEMOURS & Co, of Wilmington Del. (DuPont) offers Kapton® film (a polyimide) and Mylar ® film (a polyester). Both are believed to be suitable for use.

The base 18 is flexible or elastically deformable much like apiece of paper. That is, the base 18 may be bent or twisted or deflected upon application of a suitable force. As shown in FIG. 1, the outer element 24 of the base 18 is moved downwardly 20 a distance which is the deflection 21 by force 22 to form detent 30. Thus, the outer element 24 moves from its normal position with a non deflected length 26 to a deflected length 28 as the detent 30 is formed by the force 22. The base 18 is not elastically deformable in that it may not be pulled to vary its dimensions like a rubber band.

The insert 10 of FIG. 1 has an outer element 24 and an inner element 34 both of which are connected to the heel element 36. While the base 18 is preferably unitarily formed, it may be formed in segments or parts. For example, one or both of the outer element 24 and the inner element 34 both may be separate and be joined to the heel element 36 by suitable means such as a piece of thin tape. Of course, different pieces of the base 18 also could he joined together using suitable plastic welding procedures.

The base 18 of the insert 10 has a thickness 32 that is substantially uniform. However, the thickness 32 for the heel element 36 may be different from the thickness 32 of either the outer element or the inner element. The thickness 32 for the base 18 as shown may he from about 0.1 inch to about 0,01 of an inch. A relatively small thickness 32 is preferred for most applications in which the footwear encloses or surrounds the foot 14 (FIG. 2) like a typical shoe or boot.

The insert 10 of FIG. 1 has an outer sensor 38, an inner sensor 40 and a heel sensor 42. While three sensors are contemplated for the present invention, it should be understood that one sensor may be sufficient. Of course, in other applications two, three and even more sensors may be suitable. That is, the user may determine to use a number or quantity of sensors.

The outer sensor 38 is positioned on the outer element 24. The inner sensor 40 is positioned on the inner element 34; and the heel sensor 42 is positioned on the heel element 36. Each of the outer sensor 38, the inner sensor 40, and heel sensor 42 are formed from a material that is electrically conductive but yet has an electrical resistance that changes predictably as it is deflected. The material is preferably a conductive ink with epoxy mixture deposited in a way so that the ink deflects when the base 18 is deflected as the force 22 is applied. As the ink bends or deflects its electrical conductivity or resistance changes. As the support surface 16 is deflected to form, for example the decent 30 the material changes its resistance in value. Ohm's Law is as follows:

E=RI

Where
E equals voltage in volts
R equals resistance in ohms
I equals current in amperes.
   Thus, one can supply a voltage across the outer sensor 38, the inner sensor 40 and the heel sensor 42A and measure the resulting current through them. Alternately, one can apply a constant current and measure the resulting change in voltage. Suitable sensors to function as the outer sensor 38, the inner sensor 40 and the heel sensor 42 can be obtained from Flexpoint Sensor Systems, 106 West 12200 South, Draper, Utah 84020.

By applying an electrical signal such as a voltage or a current to any one and all of the outer sensor 38, the inner sensor 40 and the heel sensor 42, a corresponding change in the current or voltage can be detected that reflects the total amount of the deflection 21 of the outer sensor 38 and comparable deflection of the ball or inner sensor 40 and the heel sensor 42. In turn, power is supplied via conductors 44, 46, 48, 50, 52 and 54 from a power supply 56 made up of two batteries 58 and 60 wired in series. The deflection signals reflective of deflection 21 of outer sensor 38 and similar deflection signals of the inner sensor 40 and the heel sensor 42 are changes in current supplied to a converter 62. More specifically, one conductor 44, 48 and 54 is connected to the converter 62 while the other conductors 46, 50 and 52 are connected to the power supply 56. The converter 62 receives an analog electrical signal from each of the outer sensor 38, the inner sensor 40 and the heel sensor 42. The analog electrical signals are deflection signals Which are converted by the converter into digital deflection signals. The converter 62 depicted is an analog to digital converter that is a 10 bit device that operates between 10 and 1000 Hz. The operation of suitable AD converters is known and for example, is described in ABCs of ADCs (Analog to Digit al Converter Basics) by Nicolas Gray of Nov. 24, 2003.

In FIG. 2, the foot 14 is shown to have three areas of support, namely the heel area 64, the ball area (behind the big toe) 66 and the outside area (behind the little toe) 68. When upright, the user is applying a force to the support surface through each of the three areas of support on both feet. Thus, if one knew how much support or force was being applied through each area for each foot, it could suggest and, in some cases, establish if a user was properly distributing the user's weight between the user's two feet and, if not, which foot was supporting more than the other. If one knew how much support or force was being applied through the different areas of each foot, the resulting pattern could suggest and in some cases establish if the structure of the user's foot was such that the weight on that foot was being improperly distributed to one or two areas rather than traditional or typical weight distribution between the three areas. A suitably qualified person could then take steps to cause inserts for a user's shoe to redistribute the weight between feet and even areas of support in each foot.

The amount of support at each of the heel area 64, the ball area 66 and the outside area 68 may vary not only when standing statically but also when the user is moving. Information about the support or force experienced at each of the support points when moving can be useful to determine how the user is moving in relation to some standard for comparison. With the information, steps can be taken to help develop, for example, either a training program or some prosthesis (e.g., shoe insert) to help. For example, a person who is not experienced or knowledgeable about the sport of running may run in a way so that the heel of the person's running shoe strikes or impacts the running surface before the other portion of the foot. There are some who believe that it is better if the ball area 66 and possibly the outside area 68 impact the running surface before the heel area 64. Again, a training program or some prosthesis may be devised to assist the person to develop better running skills.

From FIG. 1, it can be seen that the outer sensor 38, the inner sensor 40 and the heel sensor 42 are each positioned to register with the outer area 68, the inner or ball area 66 and the heel area 64 respectively. The outer sensor 38, the inner sensor 40 and the heel sensor 42 are each shown to have a length 70, 72 and 74 respectively that is selected to extend through or substantially through the lengths 76, 78 and 80 of the outer area 68, the ball area 66 and the heel area 64 of the foot 14 (FIG. 2). The outer sensor 38, the inner sensor 40 and the heel sensor 42 may optionally be oriented to extend transverse to or normal to their present orientation. In other words, the present orientation of the sensors along the length 26 of the base 18 is preferred as the deflection 21 is more easily detectable. However, the outer sensor 38, the inner sensor 40 and the heel sensor 42 could extend in any desired orientation with each sensor in a different relative to each other.

It may also be noted that the outer sensor 38, the inner sensor 40 and the heel sensor 42 each are essentially straight. However, other shapes or forms may be used. Further, the width 82, 84 and 86 of the sensors can vary together and separately. For example, for a narrower or smaller foot, the width 82, 84 and 86 of the sensors may be less or smaller because the overall width of the user's foot 14 is much smaller.

In FIG. 1, the base 18 has a side member 90 that has a first portion 91 extends outwardly a distance 92 selected to position the crease 93 at the edge 94 of the support surface 16 either on the outer side 96 or the inner side 98 of the support surface 16. The side member 90 also has a portion 95 that extends upwardly a distance 100 comparable to the height of a shoe or to extend over the side of sandal strap. A second crease 102 allows the outer portion 104 to extend a suitable distance 106 sized in width 108 to contain the converter 62, the power supply 56, an accelerometer 110 and a transmitter 112. An ADXL 2 axis accelerometer offered by Analog Devices Inc. of Norwood, Mass. 02062 is one possible device that could be used. The accelerometer 110 supplies an analog output reflective of the acceleration of the foot 14 (FIG. 2) to the converter 62 which is then converted to a digital signal for further transmission by the transmitter 112. The batteries 58 and 60 are small cell batteries including but not limited to those sometimes loosely referred to as “watch batteries” selected to supply suitable voltage for the interconnected components shown in FIG. 1.

The support surface 16 in FIG. 1 is shown as the upper surface 16 of an insole 116 that is typically positioned inside of shoes. It has a thickness 118 that may be about ⅛ of an inch and is often made of a resilient rubber-like or neoprene-like material. It is often selected so that it allows moisture to pass there through (“breathes”) while providing suitable cushion comfort for the user. An insole 116 is typically sized to fit into a shoe or similar item of footwear. The material used for the support surface 16 varies and includes leather inserts and very rigid materials like wood. Preferably, the insole is formed of a material that is pliant and thus, has a durometer from about 10 to 20 on the Shore A scale. However, the support surface is any surface that supports a foot in connection with an item of footwear. For support surfaces which are quite rigid like wood, the illustrated outer sensor 38, an inner sensor 40 and a heel sensor 42 are not suitable because the deflection 21 will be essentially zero. In such a situation, a force sensitive resistor (see Adafruit Industries at www.adafruit.com/index) or another piezoelectric sensing device is used as a sensor rather than the flexible or deflectable outer sensor 38, inner sensor 40 and heel sensor 42 described. A force sensitive resistor can be used as one or more or all of the sensors of an insert like insert 10 with any of the insoles in selected applications as desired by the user.

The outer element 24, the inner element 34 and the heel element 36 are sized and shaped to fit into a suitable item of footwear. For example, the outer element of insert 10 has a rounded front corner 120, the inner element 34 has a rounded front 122 and the heel element has a rounded back 124 all selected to fit into a variety of footwear products. The width 126 and length 128 vary with the size of the footwear. Thus, an insert 10 for use in a size 14 EE shoe will be sized differently from one for use in a size 5 AA shoe. Also, the insertis typically fabricated by screening on the outer sensor 38, inner sensor 40 and heel sensor 42 and similarly adding the conductors 44, 46, 48, 50, 52 and 54. Thereafter a suitable coating 129 over the entire area of the insert 10 to make the insert in effect hermetically sealed so that moisture from the user's foot cannot effect the electrical performance of the insert 10 and the outer sensor 38, the inner sensor 40 and the heel sensor 42. Various liquid epoxy coatings and any suitable laminating material may be used to function as the coating 179.

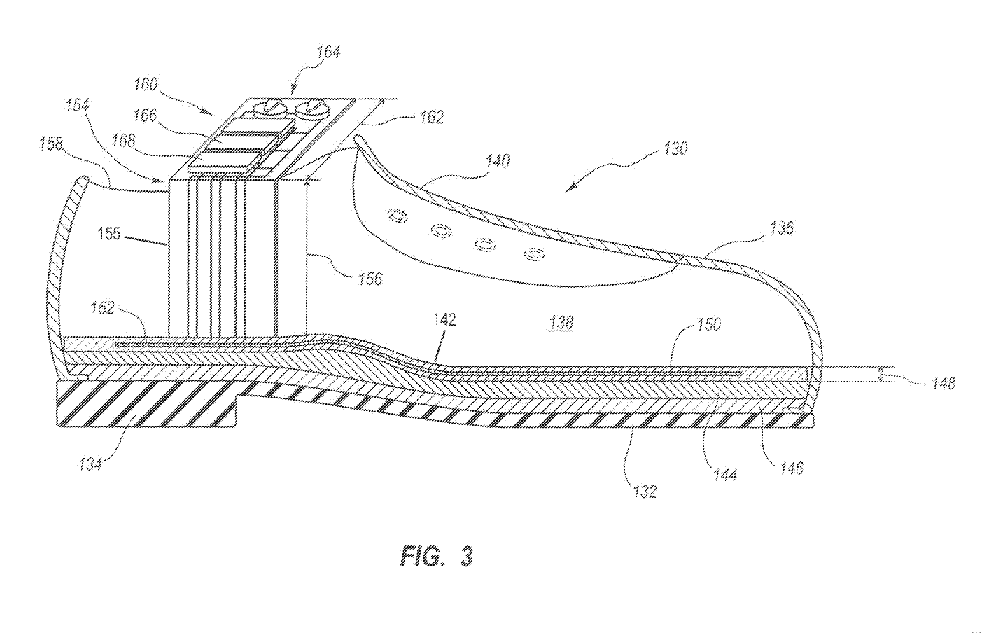

Turning now to FIG. 3, a man's shoe 130 is shown in cross section. It has a typical sole 132, heel 134 and body 136. The body 136 has aside wall 138 with a tongue 140. An insert 142 comparable to insert 10 (FIG. 1) is positioned above an insole 144 on top of a floor 146. The insert 142 is here shown to have a thickness 148 and to he formed out a resilient material. The outer sensor 150 and heel sensor 152 are shown potted in the material which is non breathing closed cell material and may be a type of epoxy material. The side member 154 has an upper portion 155 with a height 156 sized to reach the top 158 of the side wall 138. An outer portion 160 is unitarily formed with the upper portion 155 and sized in length 162 to extend less than the height 156 of the outer wall. The outer portion 160 has the power supply 164 coupled to a transmitter 166 and a converter 168. An accelerometer is not shown as it is optional.

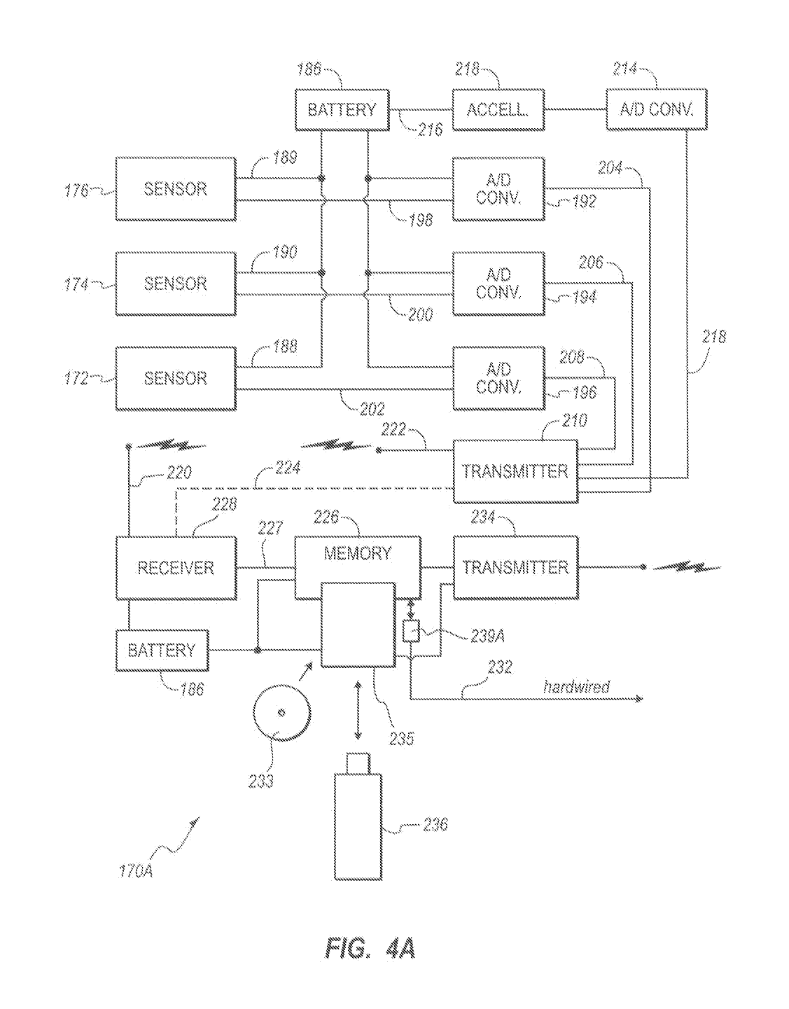

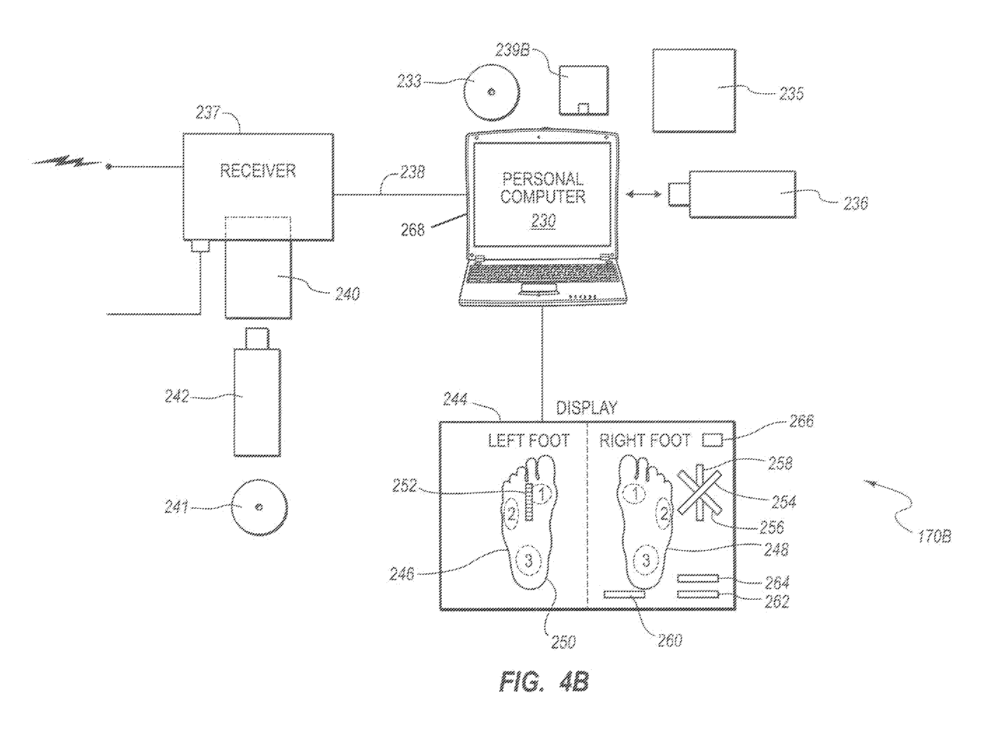

The block diagram of FIGS. 4A and 4B depicts a suitable sensing system 170A and 170B that has a plurality of sensors such as sensors 172, 174 and 176. Three sensors 172, 174 and 176 are depicted one for positioning at the ball area., the outside area and the heel area of the foot like foot 12 of FIG. 2. Of course, additional sensors can be used in other areas of the foot. For Example, FIG. 5 shows one part or element 178 of an insert like outer element 24 of base 18 having multiple sensors 180, 181, 182 and 183 oriented lengthwise (between the heel and toe of a foot) and sensors 184 and 185 oriented transverse thereto. Other configurations or patterns of sensors may be used as desired with the preferred sensors being of the type that predictably change resistance upon mechanical deflection such as the BEND SENSOR® detectors offered by Flexpoint Sensor Systems. Inc. of Draper, Utah.

The sensors 172, 174 and 176 are each connected to a power supply such as battery 186 via conductors 188, 189 and 190 as depicted in FIG. 4A. The sensors 172, 174 and 176 also are connected to analog to digital converters (AID converters) 192, 194 and 196 via conductors 198, 200 and 202. The battery 186 supplies a voltage that is applied across the sensors 172, 174 and 176 which are electrical resistors that vary in resistance as they are deflected. In turn, the electrical current in the conductors 198, 200 and 202 going to converters 192, 194 and 196 varies with the deflection. The current is in effect an analog signal that the AID converters 192, 194 and 196 convert to digital signals that are supplied via conductors 204, 206 and 208 to a transmitter 210 that processes the digital signals and transmits them as a radio frequency (RF) signal. The transmitter 210 may have a carrier and pulse or frequency modulate or it may process in any other suitable way. The digital converters preferred have a sample frequency of about 10 Hertz and an output that is supplied at a frequency that may vary from about 10 to 1000 samples or transmissions per second. In some cases, an RFID chip can be adapted as the transmitter.

FIG. 4A also shows an accelerometer 218 that is positioned on or about the foot. It could be located on the footwear, on a sock or on the lower leg. The accelerometer is a typical 3 axis device other single or two axis options may be applicable. A three axis device is used to measure the forward movement in three dimensional space of the wearer. Thus, it could be used to measure performance moving sideways or diagonally. A single axis device could measure movement in a forward/reverse direction only. The accelerometer supplies analog signals reflective of acceleration to an A/D converter 214 via conductor 216. The AID converter supplies digital signals reflective of acceleration in the X, Y and Z axis via conductor 218 to the transmitter 210 for processing and transmission as an RF signal.

The RF signal with the digital signals from the AD converters is transmitted as a low energy signal to a receiving antenna 220 that is positioned within a few feet of the transmitting antenna 222. Alternately, the RF signal may be transmitted via a suitable RF cable 224 that is sized to extend between them with sufficient length to allow full movement of the involved limb. Alternately, the digital signals can be sent by conductors, 206, 208 and 218 directly to the memory 226 for storage and further processing as described hereinafter. Inasmuch as a wire extending from the foot area to another part of the body of the user is not desired or preferred, the RF signal is transmitted from antenna 222 to antenna 220.

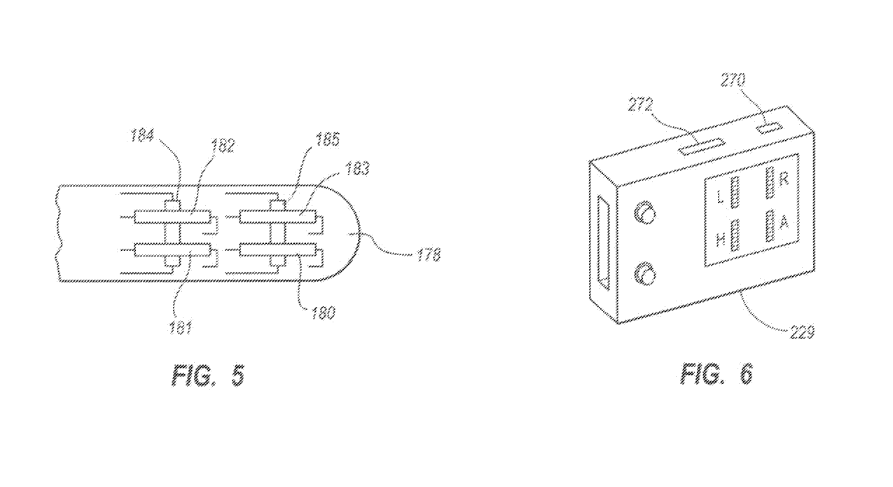

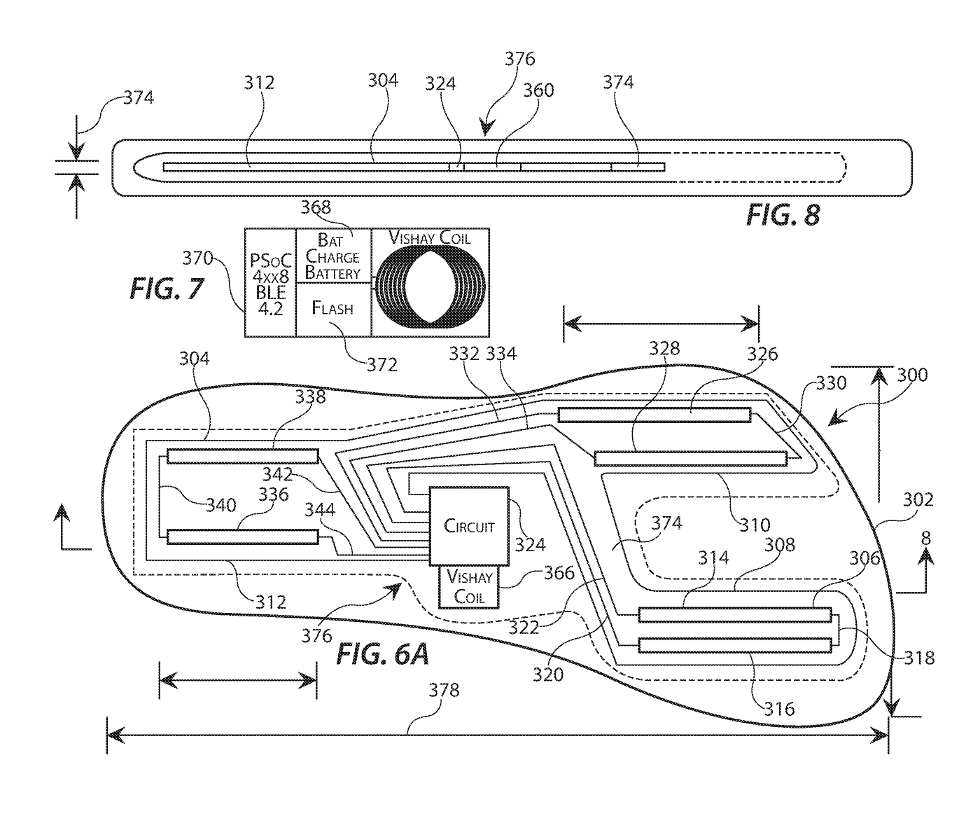

The receiver 228 is positioned in a chassis like the chassis 229 seen in FIG. 6. It receives the RF signals and processes them to extract the digital signals reflective of deflection of each of the sensors 172, 174 and 176 as well as digital signals reflective of the acceleration in up to three axes, the X axis, the Y axis and the Z axis. The digital signals are supplied to the memory 226 via conductors 227 for storage until they are delivered to a computer (PC) 230 for further processing. The digital signals may be delivered to the PC 230 by any one of several means. They may be delivered by a wire 232 which is preferred if the PC is scaled back in size and packed directly into the chassis 229. Alternately, a transmitter 234 may be configured to process and transmit all the digital signals to a suitable receiver 237 (FIG. 4B) connected to receive the digital signals and to extract them and transmit them to the PC via conductor 238.

The memory 226 may also include a suitable drive or drives to transfer the digital signals onto a CD 233, a memory chip 235 (e,g., made by SCAN DISC), and/or a flash drive 236. Of course, the CD, 234, the memo y chip 235 and the flash drive 236 may be transported to the PC 230 to deliver the digital signals thereto. Alternately, the digital signals may be delivered by a wire like wire 232 that is removably connected to suitable port 239A and 239B associated with the PC. It should be noted that the receiver 237 could also be configured to transfer the digital signals onto a suitable medium such as a memory chip 240, a CD 241 or a flash drive 242. They may be transported to a suitably configured PC for further processing.

The PC 230 is programmed or configured to process the digital signals and produce signals to present a visually perceivable display 244. As seen in FIG. 4B, the display may be an image of a left foot 246 and a right foot 248 that has the areas depicted like areas 1, 2 and 3 where sensors have been placed by use of an insert like insert 10. The display 244 may be configured to produce an image reflective of deflection if each sensor like outer sensor 38, inner sensor 40 and heel sensor 42 (FIG. 1) by selecting different colors to display for selected ranges of deflection (e.g., red equals large, yellow equals medium, black equals little) or by changing or darkening rings 250 reflective of ranges of deflection or by a scale 252 that shows deflection on a scale. The display 244 may also display acceleration in the X axis 256, the Y axis 254 and the Z axis 258 for each foot. It may also produce an image 260 depicting velocity of the user and of each foot 262 and 264 as well as an image 266 displaying the total distance traveled from the time a person starts.

The PC 230 may be scaled or sized to include the memory 226 and to fit in the chassis 229. In that event, a suitable small screen 268 is provided that can include a series of bar graphs displaying values detected by the sensors. Images can alternate between left and right foot displays every few seconds. The chassis 229 may include batteries to power all within components and be sized to be attached to the user at the waist by a suitable belt or clip. The chassis 229 may also be configured with suitable ports 270 and 272 to receive a CD or a flash drive to record digital signals for further use at a later time.

In use, a user may record all the digital signals connected to his during a particular period or event either in the PC 230 or in the memory 226 in the chassis 229. The digital signals may be compared to or with the data from and earlier or later period or event to show change or progress. This, in turn, may be used to suggest how the user may better move his or her feet to enhance his or her performance in connection with some activity. The user may learn to place more weight on the ball or the heel or to shift weight from the ball of the foot to the outside of the foot. Over time, information can be obtained and retained to show progress and help the user select exercises to improve or modify. In addition to sports and other related activity, the sensing system can be used in connection with physical therapy to monitor changes in strength and in range of motion following, for example, knee surgery and/or hip surgery and or tendon/ligament repair. In sports, it can be used to measure other foot performance values to determine corrective exercises and to compare one athlete to another or to a norm.

Turning now to a more preferred configuration, a system to measure foot function includes an insert for the left foot seen in FIG. 6A and an insert 300 for the right foot (not shown). The insert for the right foot is structured comparable to that of the one seen in FIG. 6A but reoriented to conform with the right foot of a person or user.

The insert 300 of FIG. 6A is attached to or positioned on or in a base which functions as an insole or similar structure found in many different types of footwear. That is, the insert 300 may be placed in the footwear above or on the existing insert or insole found in typical shoes or used in lieu of the existing insert or insole of the shoe. Use depends on the size of the show and the fit of the shoe to the user. The insert 300 may also be made to be the insole or part of the shoe that is in contact with the user's foot or the socks of the user when the shoe is positioned on the user's feet.

The base 302 of the insert has a sensor array 304 that is typically a substrate 306 of an eclectically insulating material such as a polyamide. The substrate should be biaxially flexible. In practice a material called Kapton® and sold by E. I. DuPont de Nemours & Co has been found suitable. The substrate is formed and sized to minimize the area of the base that it covers no the base may breathe. That is, the base 302 is typically a material that has a certain amount porosity to reduce the collection of moisture above the base 302 from perspiration during use,

The substrate 306 is formed with three sensor sections, namely a medial or big toe section 308, a lateral or little toe section 310 and a heel section 312. The medial or big toe section 208 has two detectors 314 and 316 as discussed hereinafter. Each of the two detectors are sized to be the same and are connected in series with the circuit 324 by conductors 318, 320 and 322. Similarly, the lateral or little toe detectors 326 an 328 discussed hereinafter are connected in series with the circuit 324 by conductors 330, 332 and 324. The heel section 312 also is shown with two detectors 326 and 328 that are connected in series by conductors 336, 3 8 and 340 as shown in FIG. 6A.

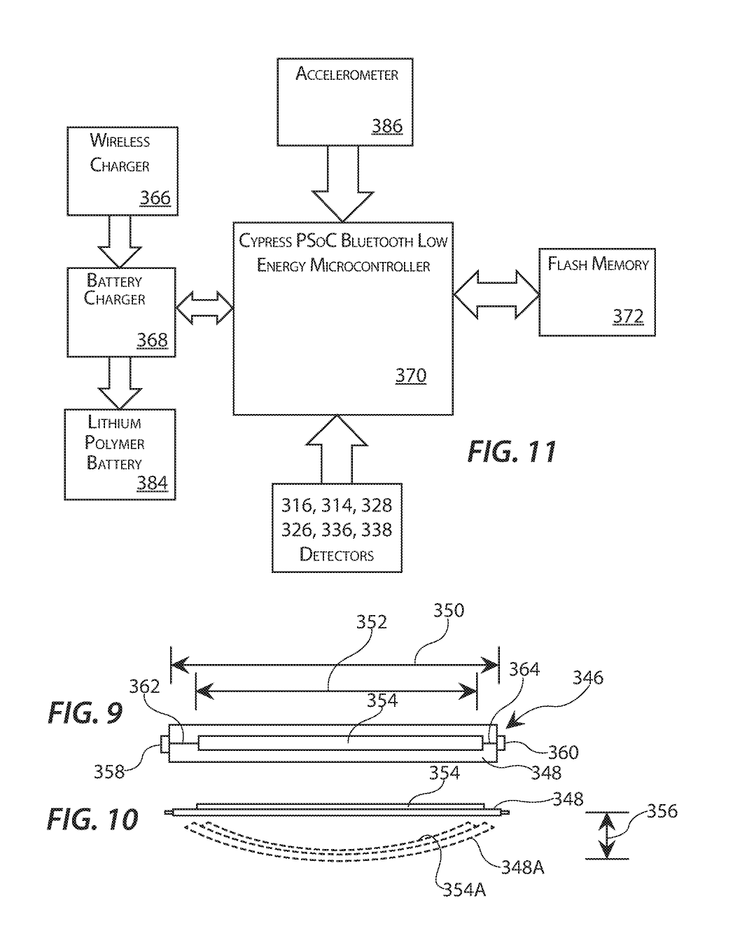

The detectors 314, 316, 326, 328, 336 and 338 are all typically sensors that change resistance upon deflection. As seen in FIG. 9, a detector 346 is formed by positioning (by, for example, silk screening) a strip of material 354 that is a combination of an epoxy and carbon on a polyamide substrate 348. As seen in FIG. 10, the substrate 348 with the material 354 deposited thereon is deflectable from the undeflected position shown in solid in FIG. 10 to a deflected position shown in dotted line. That is the substrate 348 and material 354 are movable or deflectable to positions 348A and 354A. Upon deflection, the electrical resistance of the material changes in an amount predictable by the amount 356 of deflection. The amount of deflection 356 can vary based on the user (size, weight) and the activity as well as the elasticity of the base 302 (FIG. 6A). Electrical connections 358 and 360 are positioned on the ends of the substrate 348. The electrical connections 358 and 360 are connected to the material 354 by conductors 362 and 364.

As seen in FIG. 6A, the circuit 324 has associated with it a magnetic charging device 366 which is configured to wirelessly receive electrical power and supply it to the circuit 324 to avoid use of removable batteries to supply power or even wires to a remote source of power. The circuit 324 is configured to supply electrical power to the detectors 316, 314, 326, 328, 336 and 338 and to measure the change in voltage/current which are deflection signals from each detector 316, 314, 326, 328, 336 and 338 when one or more or all of the detectors 316, 314, 326, 328, 336 and 338 are deflected. As better seen in FIG. 7, the magnetic charging device 366 is a Vishay coil available from Vishay hitertechnology of Malvern, Pa. that is connected to supply power to a battery charger 368 that is part of the circuit. The coil produces electrical energy by passing through a magnetic field spaced away from the Vishay coil. The battery charger 368 supplies power to a device to store electrical power such as a rechargeable lithium battery. The circuit 324 includes a PSoC 4XX8 BLE 4.2 microcontroller made by Cypres Semiconductor of San Jose, Calif. The microcontroller 370 includes an AD converter, a processor and a Bluetooth device. The microcontroller 370 receives the deflection signals and converts them from analog form to digital form and stores them in the flash drive 372 as discussed more fully hereinafter. The processor portion computes digital deflection signals reflective of the deflection of each of the detectors 316, 314, 326, 328, 336 and 338 and supplies digital deflection signals to the Bluetooth transmitter for transmission to a remote control device discussed hereinafter.

FIG. 8 is a side view of the base 302 of FIG. 6A along section lines 8-8. The heel section 312 is seen along with the circuit 324 and the magnetic charging device 366. The throat area 374 is also seen. The sensor array 304 is coated with a water/liquid resistant material to protect the sensor array 304 along with the magnetic charging device 366 and circuit 324. From FIG. 8, it can be seen that the circuit 324 and the magnetic charging device 366 are thin and have a height that is about the same as the sensor array 304. To minimize or even avoid discomfort to the user, the magnetic charging device 366 and the circuit 324 are positioned proximate to or to register with the instep of the user and in turn the instep area 376 of the insert 300.

Returning to FIG. 6A, it can be seen that the insert 300 has a length 378 and a width 380 which varies with shoe size so that it can fit inside of the user's shoe. Also the detectors 316, 314, 326, 328, 336 and 338 are here shown to have a length 382 to extend through the deflection area. However, in selected applications the length 382 of each detector 316, 314, 326, 328, 336 and 338 can be different.

In FIG. 11 it can be seen that the microcontroller 370 is connected to the detectors 316, 314, 326, 328, 336 and 338 to receive analog deflection signals. The microcontroller 370 converts them to a digital form and stores them as needed in the flash memory 372. The microcontroller 370 includes a Bluetooth function and transmits digital deflection signals to a remote device as discussed hereinafter. The microcontroller 370 is powered by a lithium polymer battery 384 which is charged by an associated battery charger 368 that is powered by a wireless magnetic charging device 366. An accelerometer 386 may be placed on the circuit 324 to supply analog acceleration signals to the microcontroller 370 which converts them to digital form. The Bluetooth transmitter of the microcontroller 370 transmits the digital acceleration signals to a remote device as discussed hereinafter.

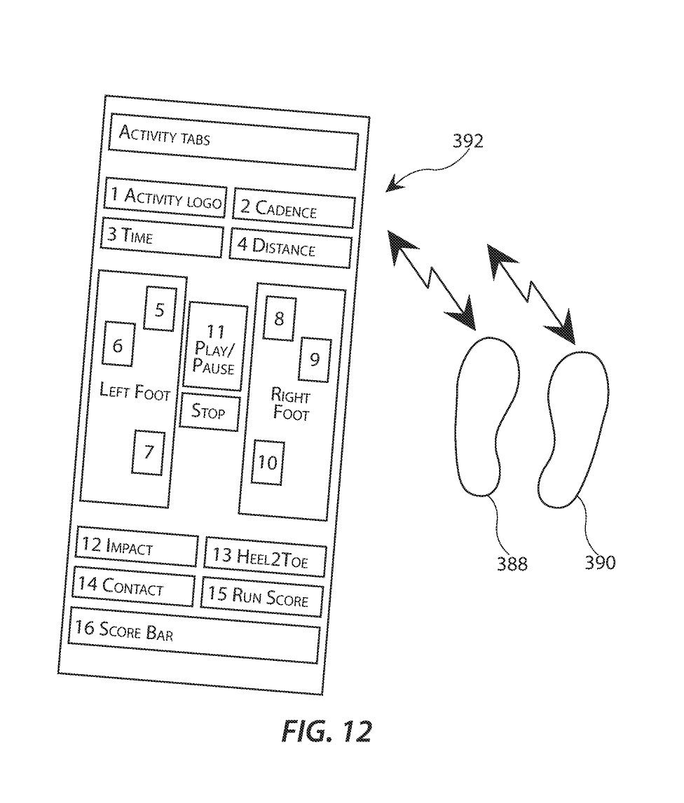

In FIG. 12, a left insert 388 and a right insert 390 are comparable to the insert 300 depicted in FIG. 6A. The inserts transmit by Bluetooth digital deflection signals and digital acceleration signals to a remote control device 392 which is depicted as a cell phone now available from a variety of manufacturers (e.g., Apple. Inc., Samsung; and others) and programmed with an application or “app” so that the cell phone may be used to control the system and display data received from the left insert 388 and the right insert 390. Various tablets or pads may he used as well including an iPad offered by Apple. Inc. So long as the device has Bluetooth and is capable of being programmed with an “app”, it should he available to function as the control device as herein discussed.

As seen in FIG. 12, the device 392 has an activity tab which is to be operated to change the activity from running to some other sport. When the activity tab is operated to select running as a sport, the activity selected can be varied by operating the activity logo. For example, for running, the “app” is configured to operate in three modes: idle mode, play mode and pause mode. It may also be turned off by turning off the phone or other device.

In the idle mode, the user can view real time values of the detectors 316, 314, 326, 328, 336 and 338 to confirm that the system is working In a selected configuration, the insert sensors are depicted on the screen of the control device 392 as a circle that grows in size and changes color based on the values of the digital deflection signals. Generally values close to zero will have a small circle and be green; and values that are large will have a large circle and be red. All other values on the screen will be empty or not displayed.

When operating the Activity logo to operate the control device 392 in the play mode, a number of different values will be displayed for the involved activity such as “running.” The various measurements and calculations presently available are as set forth in the following table.

TABLE 1

| Number | Data | |
|---|---|---|
| 1 | Activity Logo | Displays selected activity |
| 2 | Cadence | Steps per unit time |
| 3 | Time | Time since start |
| 4 | Distance | Distance from start |
| 5 | Left medial load | Percentage of total load |
| 6 | Left lateral load | Percentage of total load |
| 7 | Left heel load | Percentage of total load |
| 8 | Right medial load | Percentage of total load |
| 9 | Right lateral load | Percentage of total load |
| 10 | Right heel load | Percentage of total load |
| 11 | Pause | Transition from play to pause |
| 12 | Impact force | How hard is user landing |
| 13 | Heel to toe (time between) | Time from heel touch to toe |
| 14 | Contact time | How long foot in contact |
| 15 | Run score | A number calculated based on |
| | | value assigned to each task |
| 16 | Score display | Visualize run score |

The “app” may he configured so that the score of a particular metric may be green if it is above a set threshold like 90%. If the score is between 75% and 90%, the screen is chartreuse. If the score is between 50% and 75%, then the screen is yellow. If it is between 25% and 50% the screen will be orange, and if 25% and bellow, the screen will be red. While the coloring preferably applies to all but the load values (items 5 through 10) where the sensor color is green if below 40% and chartreuse if between 40% and 49%. It is yellow if between 49% and 65% and orange between 66% and 82%. It is red if above 82%. In normal operations, all data is saved to the file every 15 seconds. After operation, the data will be automatically uploaded to the cloud if the system has been configured to do so.

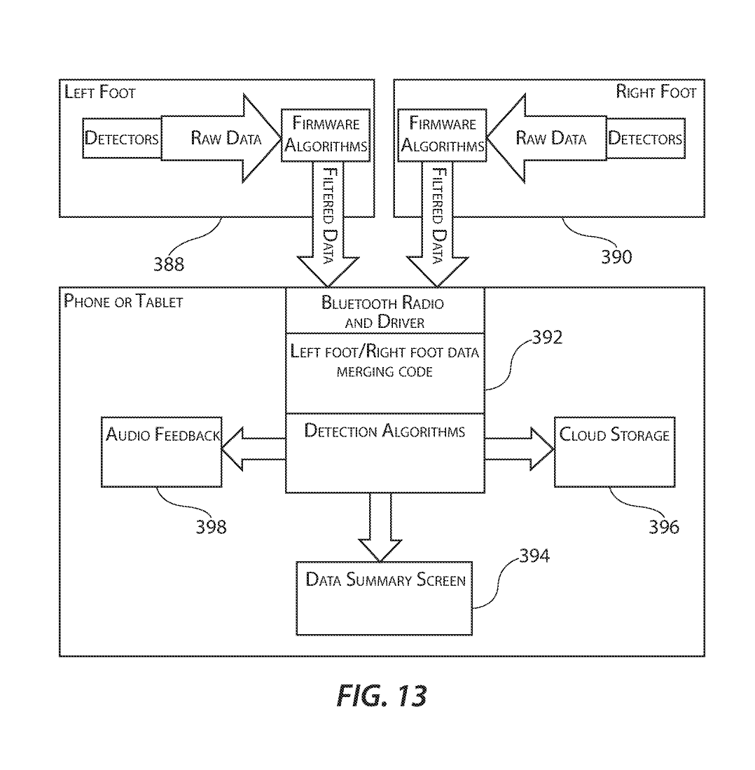

Turning to FIG. 13, the basic architecture of the “app” seen in the control device 392 is illustrated. The detectors of each of the left foot 388 and the right foot 390 process through the microcomputer 370 using firmware algorithms for interaction with the Bluetooth receiver in the control device 392. The data is calculated and presented on screens 394, stored in cloud storage and announced to the user with an audio signal 398.

In operation, the user places the left insert 388 and right insert 390 into a shoe and performs an activity while monitoring the data obtained on the screens of the control device 392. When not in use, the user may place the left insert 388 and the right insert 390 over a charging coil to wirelessly charge the battery 384.

Of course with data available from the display, the user may make adjustments to running techniques to improve performance.

Those skilled in the art will recognize that many changes or variations may be made to the above illustrated system and the components thereof without departing from the teachings set forth herein. Therefore, the details of the embodiment or alternatives illustrated and/or described are not intended to limit the scope of the appended claims.

1. A sensing system for use with footwear, said sensing system comprising:
an insert for placement between the support surface of an item of footwear and the foot of a user;
at least one sensor associated with said insert and positioned to deflect upon force applied h the foot of a user, said sensor being configured to transmit detection signals reflective of the deflection of said at least one sensor;
a circuit connected to said at least one sensor to receive said detection signals from said at least, one sensor, said circuit including
an analog to digital converter to convert said detection signals to digital deflection signals,
a flash memory to retain digital detection signals,
a processor to process said digital detection signals and generate sensed deflection signals reflective of said detection signals of said at least one sensor and to supply a sensed deflection signal to
a transmitter connected to said processor and configured to wirelessly transmit said sensed detection signals, and a
power storage structure to receive and store power and to supply electrical power to the circuit;
a power supply to wirelessly receive power from a remote source and supply said power to said power storage structure;
a control device positioned remotely from said at least one sensor, said control device being configured to wirelessly receive said sensed detection signals, said control device having
a screen to display images reflective of said sensed detection signals,
a processor to process said sensed detection signals in accordance with functions selected by an operator,
controls for operation by the operator to select functions for processing the sensed detection signals, said controls including a function to visually display on said control device images reflective of said sensed detection signals.

2. The sensing system of claim 1 wherein said one sensor is positioned proximate a support point of said user's foot.

3. The sensing system of claim 1 wherein at least one sensor is a plurality of sensors each positioned proximate a different support point of said user's foot.

4. The sensing system of claim 2 wherein said insert includes three sensors each positioned proximate each of three support points of a user's foot.

5. The sensing system of claim 4 wherein each of said three sensors includes a substrate with a resistance material deposited thereon which resistance material predictably changes its electrical resistance upon deflection.

6. The sensing system of claim 1 wherein said insert is encased in a water resistant material.

7. The sensing system of claim 1 further including an accelerometer attached to said insert, said accelerometer being configured to sense the acceleration of the insert as it is moved by the user and to supply acceleration signals to said circuit, wherein said circuit is configured to receive said acceleration signals, to store said acceleration signals and to supply digital acceleration signals reflective of said acceleration signals, wherein said transmitter is configured to transmit said digital acceleration signals and wherein said control device is configured to receive said digital acceleration signals and to generate a display reflective of the acceleration upon operation of a selected control of said control device detected by said accelerometer.

8. The sensing system of claim 1 wherein said control device includes a receiver configured to receive said sensed detection signals and to supply said sensed detection signals to said processor.

9. The sensing system of claim 8 wherein said processor is configured to process said sensed detection signals to a cloud storage system.

10. The sensing system of claim l wherein said control device includes an audio device and wherein said processor is configured to supply audio signals reflective of said sensed detection signals.

11. The sensing system of claim I wherein the circuit is formed on a wafer thin substrate and wherein the power supply is a thin coil configured to receive power magnetically.

12. The sensing system of claim 11 wherein the wafer thin substrate is sized to fit under the arch of a foot.
